(19) United States
(12) Patent Application Publication
(10) Pub. No.: US 2008/0038446 A1
(43) Pub. Date: Feb. 14, 2008

(54) METHOD OF COATING STENT WITH GLYCOPROTEIN RECEPTOR INHIBITOR

(76) Inventors: Myung-Ho Jeong, Seo-Gu (KR); Dong Lyun Cho, Buk-Gu (KR)

Correspondence Address: CHA & REITER, LLC, 210 ROUTE 4 EAST STE 103, PARAMUS, NJ, 07652, US

(21) Appl. No.: 11/744,919

(22) Filed: May 7, 2007

(30) Foreign Application Priority Data
May 9, 2006 (KR) 10-2006-0041325

(51) Int. Cl. A61L 33/00

(52) U.S. Cl. 427/2.25

(57) ABSTRACT

A method of coating a surface of a stent with abcixmab (Reopro) which is one of glycoprotein IIb/IIIa receptor inhibitors is provided. In case of acute coronary occlusion, the stent made of a stainless steel treated with carbon is coated with the Reopro having an antiplatelet action against platelets, thereby remarkably preventing the stent restenosis. The coating method includes activating the surface through cleaning and plasma process, and coating the surface with a thin film having an amine radical. A process condition suitable for the feature of the carbon is induced at each process.

### BACKGROUND OF THE INVENTION

1. Field of the Invention

The present invention relates to a method of coating a stent which is inserted into narrowed or occluded blood vessels of a human body to vasodilate the blood vessel, with an anti-platelet agent, and more particularly, to a method of coating a surface of a stent with abcixmab (hereinafter referred to as “Reopro” (trademark)) which is one of glycoprotein IIb/IIIa receptor inhibitors, so as to inhibit the stent restenosis after stenting operation, in case that the stent is made of a stainless steel treated with carbon.

2. Background of the Related Art

In general, if blood vessels, such as coronary artery, peripheral vascular system, and so forth, are occluded due to deposition of cholesterol or the like, it blocks the flow of blood to induce a fatal disease.

In particular, a heart is the most major organ to control the flow of blood in a human body. If a problem arises in the cardiac blood vessel, it is difficult to control the flow of blood which supplies the blood to all parts of the human body, and thus, it induces a serious problem.

In case of a patient suffering from angina pectoris, for example, the patent feels a sever pain, or may suddenly die.

In case of the coronal occlusion of a heart, if the occlusion is not settled within about 90 minutes, a patient may die or may be suffered from a brain injury.

In this instance, stenting operation using a stent is widely conducted recently, rather than conventional methods such as phlebotomy, arteriotomy, medication of medicines to dissolving thrombus, and so forth.

More specifically, a guide catheter is inserted in a blood vessel through an inlet port of the blood vessel, and is traveled to a lesion in the blood vessel, and then a guide wire is inserted in the guide catheter positioned proximity to the lesion. The guide wire is guided by the guide catheter, and is positioned at the curved lesion of the blood vessel (see FIG. 3a). After the guide wire is inserted in a bladder catheter, the bladder catheter is moved to the lesion of the blood vessel, and a bladder of the bladder catheter is positioned at the lesion (see FIG. 3b). Finally, the bladder is inflated by supplying air to the bladder with a pressing means coupled to a connector of the bladder catheter, so that the lesion is expanded, with a blocking substance being compressed (see FIG. 3c).

Since the flexible bladder made of nylon material has a limit to compression of the blocking substance in the blood vessel, a stent made of a stainless steel mesh is mounted in the bladder. When the bladder is inflated, the stent is also expanded to increase a compressing force against the blocking substance and also decrease coagulating action of blood on a surface of the stent due to Reopro coated on the surface of the stent.

The stent is left in the blood vessel after the stenting operation. In case that the surface of the stent is not smooth, some problems such as blood coagulation may occur. In order to solve the above problem, after the stent is subjected to polishing and annealing, the stent is coated with carbon under conditions of a high pressure, there by converting a coarse polished surface into a smooth coated surface. Also, since the stainless steel stent coated with the carbon should be further coated with Reopro, there is required for a suitable coating method.

### SUMMARY OF THE INVENTION

Accordingly, the present invention is directed to a method of coating a stent with Reopro that substantially obviates one or more problems due to limitations and disadvantages of the related art.

An object of the present invention is to provide a method of coating a surface of a stent with abcixmab (Reopro) which is one of glycoprotein IIb/IIIa receptor inhibitors, in which the stent made of a stainless steel treated with carbon is coated with the Reopro having an antiplatelet action against platelets, thereby preventing the platelets from being coagulated on the surface of the stent by the action of the antiplatelet agent coated on the stent and also preventing the coagulating action of blood on the surface of the stent.

To achieve the object and other advantages, according to one aspect of the present invention, there is provided a method of coating a surface of a stent with a platelet glycoprotein receptor inhibitor (Reopro), in which the stent is made of stainless steel implanted with carbon, comprising a wet cleaning process of eliminating alien substances from the surface of the stent; a surface activating process of activating the surface of the stent, in which after the cleaned stent is fixed to a tubular reactor made of a Pyrex glass tube, an internal pressure of the reactor is lowered by atmospheric pressure of up to 0.01, and oxygen, argon, or a mixture gas of argon and hydrogen is introduced into the reactor and a discharge power of 5 to 200 W is applied to electrodes of the reactor during 1 to 30 minutes to generate a plasma in the reactor; a coating process of coating the surface of the stent with a thin film having an amine radical, in which after the surface activating process is completed, the internal pressure of the reactor is lowered by atmospheric pressure of up to 0.01, and diaminocyclo hexane (DACH) monomer is introduced into the reactor and a discharge power of 50 to 100 W is applied to the electrodes during 1 to 30 minutes to generate a plasma in the reactor; and a grafting process of grafting the stent with the Reopro, in which after the coating process is completed, the stent is withdrawn at air, and is immersed in a Reopro solution containing Reopro, sodium citrate, and cyamide at a temperature of 25 to 50° C. during 5 minutes to 80 minutes.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a diagram schematically depicting a coating process according to the present invention;

FIG. 2 is a perspective view of a vascular stent according to the present invention; and

FIG. 3 is a view illustrating a process of expanding a blood vessel with a general stent.

### DETAILED DESCRIPTION OF THE PREFERRED EMBODIMENTS

A preferred embodiment according to the present invention will now be explained with reference to the accompanying drawings.

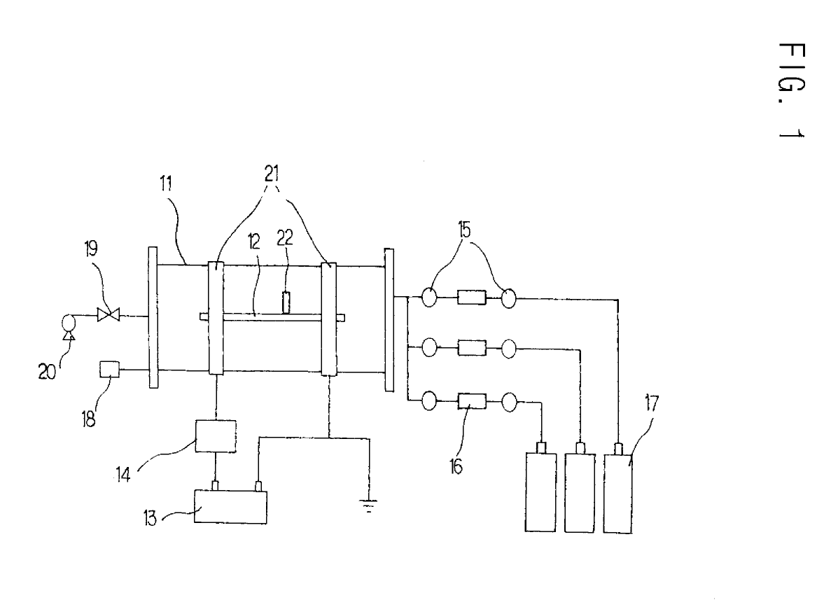

Referring to FIG. 1 showing a coating apparatus of the present invention, the coating apparatus includes a tubular reactor 11 made of a Pyrex glass tube, electrodes 21 installed on left and right sides of the reactor 1, an RF power supply source 13 for supplying an RF power to the electrodes, a pressure gauge 18 connected to the reactor 11 for controlling a pressure of the reactor, a vacuum pump 20 connected to the reactor 11 for controlling vacuum of the reactor using a valve 19, a stop valve 15 connected to the reactor 11 for controlling supply of a gas or liquid from a gas or liquid container 17, and a flow rate meter 16.

A sample support plate 12 is fixed to an inner portion of the reactor, and a stent is positioned on the sample support plate 12.

The coating apparatus illustrates one example of the present invention, and does not limit the scope of the present invention. Of course, an alteration or modification of a part of the coating apparatus to conduct a Reopro coating process is contained in the scope of the present invention.

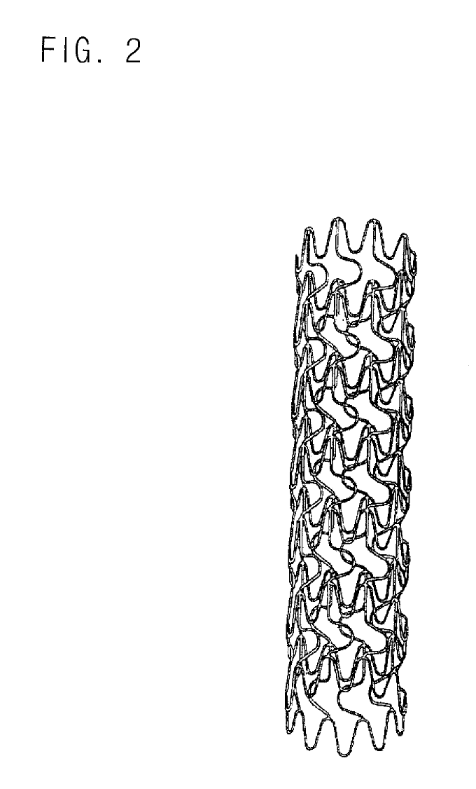

FIG. 2 is a perspective view of a vascular stent coated with carbon according to the present invention. The stent is made of a mesh of stainless steel to have resilience and flexibility. Also, the stent has a feature of maintaining its shape.

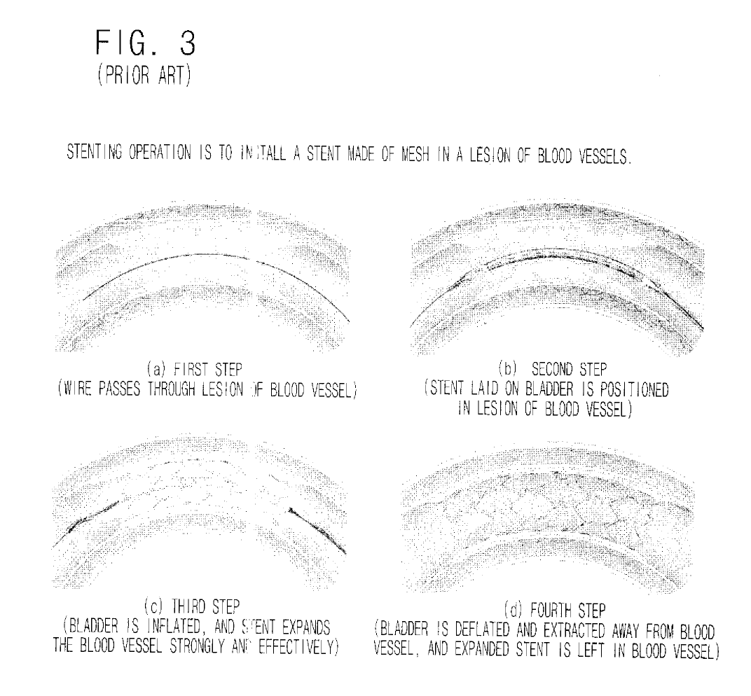

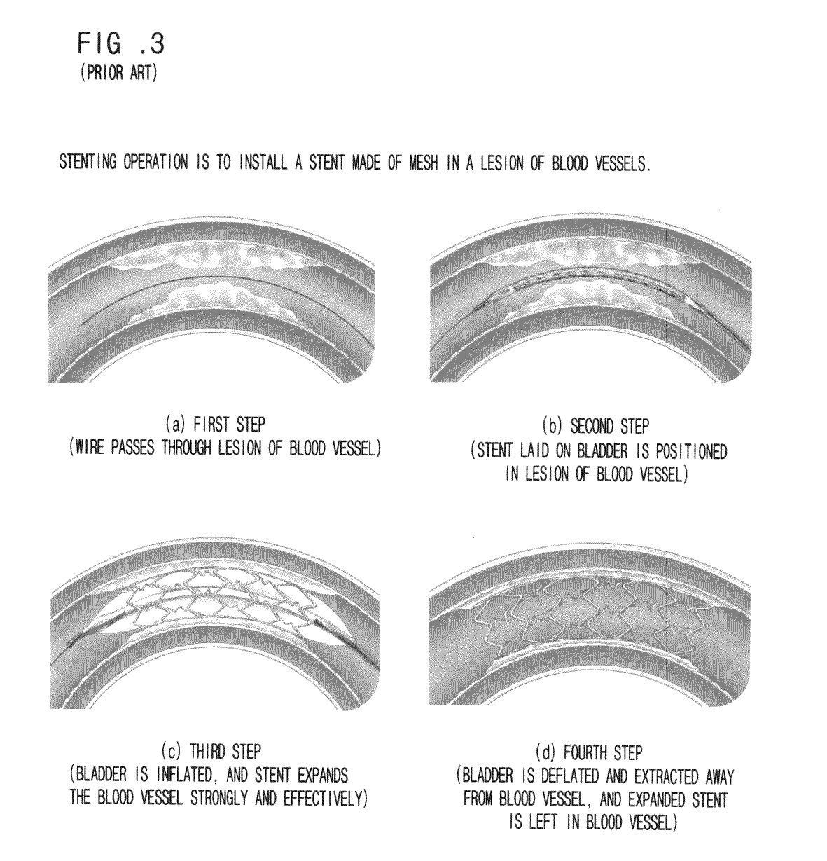

FIG. 3 is a view illustrating a process of expanding a blood vessel with a general stent.

FIG. 3a shows a first step that a wire passes through a lesion of the blood vessel. FIG. 3b shows a second step that a stent laid on a bladder is positioned in the lesion of the blood vessel. FIG. 3c shows a third step that the bladder is inflated, and the stent expands the blood vessel strongly and effectively. FIG. 3d shows a fourth step that the bladder is deflated and extracted away from the blood vessel, and the expanded stent is left in the blood vessel.

A method of coating the stainless steel implanted with carbon with Reopro will now be described with reference to the drawings.

Cleaning Process

A stent 22 is subjected to a wet cleaning to eliminate alien substances from a surface of the stent. For example, the stainless steel stent 22 implanted with carbon is immersed in a solution mixed with water and alcohol at a ratio of 1:1, and then is cleaned by use of an ultrasonic cleaning machine during 5 minutes or more.

Surface Activating Process

After the cleaning process is completed, the stent 22 is treated by plasma to eliminate molecular alien substances from the stent and simultaneously activate the surface of the stent 22 implanted with carbon. To the end, after the cleaned stent 22 is fixed to the tubular reactor 11 made of Pyrex glass tube, the internal pressure of the reactor 11 is lowered by atmospheric pressure of up to 0.01 by use of the vacuum pump 20, and oxygen, argon, or a mixture gas of argon and hydrogen is introduced into the reactor 11 to maintain the pressure at 0.05 to 5.0 torr.

The plasma is generated in the radiator by applying a discharge power of 5 to 200 W to the electrodes from the RF power supply source 13 during 1 to 30 minutes.

It is preferable that the discharge power is 50 W or more and the processing time is 10 minutes or more, in order to sufficiently clean and activate the surface of the stent 22.

Coating Process

After the surface activating process is completed, the internal pressure of the reactor is lowered by atmospheric pressure of up to 0.01, and diaminocyclo hexane (DACH) monomer is introduced into the reactor 11 to maintain the reactor 11 at atmospheric pressure of 0.05 to 1.0.

The plasma is generated in the reactor by applying a discharge power of 50 to 100 W to the electrodes from the RF power supply source 13 during 1 to 30 minutes, preferably 15 to 20 minutes.

Introduction of an amine radical is preferable above 15 minutes, but a thin film to be coated may be too thick if it is performed during above 20 minutes. In this instance, the internal stress in highly increased, and thus the coated thin film may be peeled off from the surface of the stent 22.

It is preferable that the discharge power to generating the plasma is 40 to 60 W. The reason can be understood from the spectrum of the thin film which is analyzed by FTIR/ATR, as shown in Table 1.

Graph 1

[image]

In Graph 1, a curve (a) indicates a surface of a stainless steel, a curve (b) indicates a surface of stainless steel treated by DACH plasma, and a curve (c) indicates a surface of a stainless steel treated by DACH plasma and then cleaned by water. It would be understood that an amine radical (1630 cm⁻¹ and 3300 cm⁻¹) is generated in the curve (b) and the amine radical is still existed in the curve (c).

[image]

[image]

[image]

Graphs 2 and 3 show variations of spectrums according to the discharge power before and after the water cleaning. It would be understood that when the discharge power is 40 to 60 W, an intensity of a feature peak of an amine radical appeared at 1630 cm⁻¹ and 3300 cm⁻¹ is the strongest.

Grafting Process

The coating process is completed, the stent 22 implanted with the amine radical is withdrawn at the air, and is immersed in a Reopro solution containing Reopro, sodium citrate, and cyamide at a temperature of 25 to 50° C. during 5 minutes to 80 minutes to graft the stent with the Reopro. In order to eliminate the Reopro weakly adhered on the stent, the stent is cleaned with a deionized water during 1 to 5 minutes.

With the above description, according to the present invention, the surface of the stainless steel stent implanted with carbon is coated with the Reopro, thereby inhibiting coagulation of platelets onto the surface of the stent left in the blood vessel after the stenting operation, and thus preventing the stent restenosis.

The forgoing embodiment is merely exemplary and is not to be construed as limiting the present invention. The present teachings can be readily applied to other types of apparatus. The description of the present invention is intended to be illustrative, and not to limit the scope of the claims. Many alternatives, modifications, and variations will be apparent to those skilled in the art.

1. A method of coating a surface of a stent with a platelet glycoprotein receptor inhibitor (Reopro), in which the stent is made of stainless steel implanted with carbon, comprising
a wet cleaning process of eliminating alien substances from the surface of the stent;
a surface activating process of activating the surface of the stent, in which after the cleaned stent is fixed to a tubular reactor made of a Pyrex glass tube, an internal pressure of the reactor is lowered by atmospheric pressure of up to 0.01, and oxygen, argon, or a mixture gas of argon and hydrogen is introduced into the reactor and a discharge power of 5 to 200 W is applied to electrodes of the reactor during 1 to 30 minutes to generate a plasma in the reactor;
a coating process of coating the surface of the stent with a thin film having an amine radical, in which after the surface activating process is completed, the internal pressure of the reactor is lowered by atmospheric pressure of up to 0.01, and diaminocyclo hexane (DACH) monomer is introduced into the reactor and a discharge power of 50 to 100 W is applied to the electrodes during 1 to 30 minutes to generate a plasma in the reactor; and
a grafting process of grafting the stent with the Reopro, in which after the coating process is completed, the stent is withdrawn at air, and is immersed in a Reopro solution containing Reopro, sodium citrate, and cyamide at a temperature of 25 to 50° C. during 5 minutes to 80 minutes.

2. The method as claimed in claim 1, wherein the discharge power for generate the plasma in the coating process is 40 W to 60 W.

3. The method as claimed in claim 1, wherein a processing time of the coating process is 15 minutes to 20 minutes.

4. The method as claimed in claim 1, wherein in the surface activating process, the discharge power to generate the plasma is 50 W or above, and a processing time is 10 minutes or above.

5. The method as claimed in claim 2, wherein a processing time of the coating process is 15 minutes to 20 minutes.
